(19) United States
(12) Patent Application Publication
(10) Pub. No.: US 2024/0293315 A1
(43) Pub. Date: Sep. 5, 2024

(54) CARBON DIOXIDE-GENERATING NANOMATERIAL

(71) Applicant: IUCF-HYU (INDUSTRY-UNIVERSITY COOPERATION FOUNDATION HANYANG UNIVERSITY), Seoul (KR)

(72) Inventors: Kuen Yong LEE, Seoul (KR); Chunggoo KIM, Incheon (KR)

(21) Appl. No.: 18/572,792

(22) PCT Filed: Jun. 21, 2022
(86) PCT No.: PCT/KR2022/008764

§ 371 Date: Dec. 21, 2023

(30) Foreign Application Priority Data
Jun. 24, 2021 (KR) 10-2021-0082419

(51) Int. Cl. A61K 9/107; A61K 9/00; A61K 9/46; A61K 33/00; A61K 47/10; A61K 47/42; A61P 3/04

(52) U.S. Cl. CPC A61K 9/107; A61K 9/0007; A61K 9/0019; A61K 33/00; A61K 47/10; A61K 47/42; A61P 3/04; A61K 2800/222

(57) ABSTRACT

The present invention relates to a carbon dioxide-generating micelle. In the present invention, a micelle which uses a material with high biocompatibility and has a multi-arm structure capable of generating a large amount of carbon dioxide is produced, and thus, a greater amount of carbon dioxide can be generated, as compared to conventional polyethylene glycol-based micelles. In addition, in the present invention, intracellular penetration is improved by using a ligand (peptide), and thus, it is possible to target adipocytes and minimize effects on surrounding tissues and cells. The carbon dioxide-generating micelle according to the present invention can be produced as an injectable preparation, and can be applied to local lipolysis supplements or diet and beauty products that break down localized fat.

### TECHNICAL FIELD

The present invention relates to a carbon dioxide-generating nanomaterial.

### BACKGROUND ART

Reducing the subcutaneous fat under the epidermis and dermis of the skin is one of the most important areas in cosmetic treatment, and various procedures are used for such cosmetic treatment.

Procedures for reducing subcutaneous fat include: liposuction, which involves inserting a cannula into the subcutaneous fat and suctioning to remove fat; cryotherapy, which involves attaching a cooling pad to the skin to freeze and eliminate the subcutaneous fat; a thermal heating procedure, which involves heating the subcutaneous fat through irradiation with high frequency or ultrasound waves to remove it; carboxytherapy, which involves slowly injecting carbon dioxide (CO₂) into the subcutaneous fat with a fine needle to remove fat by promoting the flow of blood and lymphatic circulation in fat tissue; and mesotherapy, which involves injecting drugs for treating obesity into the subcutaneous fat.

Among these procedures, liposuction is known to be the most effective procedure but has disadvantages in terms of pain accompanying the procedure and post-operative care. For example, liposuction can cause bleeding in suctioning fat, and pain accompanies the procedure. The pain can last even after the procedure, and in some cases, patients have to take a painkiller. In addition, it is recommended to wear compression garments more than one week after liposuction, and post-operative care is required for about one month after the procedure.

Cryotherapy is a simple procedure but is less effective. Korean Unexamined Patent Publication No. 10-2011-0119640 used an invasive procedure in which a probe, which is cooled by a refrigerant circulating inside, is inserted into subcutaneous fat. Although the invasive cryotherapy can shorten the procedure time compared to the non-invasive cryotherapy, it requires a fairly long procedure time to prevent the necrosis of subcutaneous fat due to the freezing.

Carboxytherapy is a procedure that intensively treats areas where fat is excessively accumulated, and Korean Registered Patent No. 10-0772961 improved fat removal efficiency by performing mesotherapy and carboxytherapy. However, the patent uses two separate needles for the two procedures, so the invention has disadvantages such as a complicated interior structure and two separate incisions.

Currently, local lipolytic supplements approved by the Ministry of Food and Drug Safety (MFDS) have very limited uses, and off-label procedures are frequently carried out. These off-label procedures lack evidence of safety and efficacy and are in the blind spot of safe use management because they are not covered, and systemic management is insufficient.

One of the MFDS-approved lipolytic supplements is Belkyra, which eliminates fat cells by destroying the cell membrane of localized fat cells. However, Belkyra is used only for double chin procedures. In addition, it is currently reported that the risk of breast cancer or colon cancer increases when the drug is used because it non-specifically destroys cell membranes and thus has a significant effect not only on adipocytes but also on the surrounding cells, causing side effects in the surrounding cells.

### DISCLOSURE

### Technical Problem

The present invention is directed to providing a lipolytic supplement that breaks down localized fat or a cosmetic product for weight loss, which can be produced as an injectable preparation.

In addition, the present invention is directed to providing a polyethylene glycol-based micelle, which can generate a large amount of carbon dioxide and to which a peptide is conjugated to improve cell penetration.

### Technical Solution

One aspect of the present invention provides a carbon dioxide-generating micelle including one or more compounds selected from the group consisting of a compound represented by Chemical Formula 1 below and a compound represented by Chemical Formula 2 below:

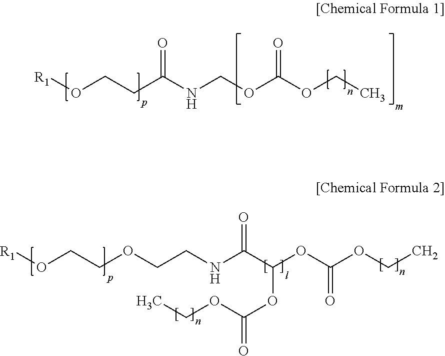

in Chemical Formula 1 or Chemical Formula 2,
R₁ represents hydrogen, a C₁ to C₅ alkyl group, an amine group, a C₁ to C₅ alkylamine group, a carboxyl group, or a C₁ to C₅ alkylcarboxyl group,
p is an integer of 12 to 227,
n is an integer of 2 to 14,
m is an integer of 1 to 3, and
l is an integer of 4.

Another aspect of the present invention provides a method of preparing a carbon dioxide-generating micelle, including: synthesizing a hydroxylated polyethylene glycol derivative, which is one or more compounds selected from the group consisting of the compound represented by Chemical Formula 1 and the compound represented by Chemical Formula 2, by mixing hydroxylated polyethylene glycol and an alkyl chloroformate; and
synthesizing a micelle by dissolving the hydroxylated polyethylene glycol derivative in one or a mixed solvent of two or more selected from acetonitrile, methylene chloride, chloroform, and methanol and evaporating the solvent.

Still another aspect of the present invention provides a composition for fat reduction, including the above-described carbon dioxide-generating micelle.

### Advantageous Effects

A carbon dioxide-generating micelle according to the present invention can break down fat by killing fat cells by being locally injected and deposited in fat cells to generate carbon dioxide.

In particular, the present invention uses materials with high biocompatibility and prepares a micelle with a multi-arm structure, which can generate a large amount of carbon dioxide, making it possible to generate much more carbon dioxide compared to an existing polyethylene glycol-based micelle.

In addition, in the present invention, cell penetration is improved by using a ligand (peptide), so it is possible to target fat cells and minimize an adverse effect on surrounding tissue and cells. Therefore, it is possible to minimize side effects of drugs and develop products for safer procedures. The micelle according to the present invention can be applied to specific areas of the body such as the chin, thighs, arms, and belly, which are frequently treated.

The carbon dioxide-generating micelle according to the present invention can be produced as an injectable preparation and used for cosmetic treatment for weight loss and obesity treatment such as a lipolytic supplement to break down localized fat, a formulation corrector, or a cosmetic product for weight loss.

### DESCRIPTION OF DRAWINGS

FIG. 1 is a schematic diagram of the accumulation of carbon dioxide-generating micelles in fat cells and a reduction in fat through gas generation according to an example of the present invention.

FIG. 2 illustrates photographs of hydroxylated polyethylene glycol derivatives prepared according to an example of the present invention, depending on a gas generation time.

FIG. 3 illustrates a graph showing results of cytotoxicity evaluation of peptide-conjugated micelles prepared according to an example of the present invention.

FIG. 4 illustrates graphs showing evaluation results of the fat cell killing effect of gas non-generating groups.

FIG. 5 illustrates graphs showing evaluation results of the fat cell killing effect according to a micelle concentration and a peptide combination.

FIG. 6 illustrates a graph showing evaluation results of the in vivo efficacy of micelles prepared according to an example of the present invention.

FIG. 7 illustrates photographs of stained tissue where micelles prepared in an example of the present invention are injected.

FIG. 8 illustrates photographs and graphs showing the retention of peptide-conjugated micelles prepared in an example of the present invention.

### BEST MODES OF THE INVENTION

Hereinafter, a carbon dioxide-generating micelle of the present invention will be described in further detail.

A carbon dioxide-generating micelle (hereinafter “micelle”) according to the present invention includes one or more compounds selected from the group consisting of a compound represented by Chemical Formula 1 below (hereinafter “compound of Chemical Formula 1”) and a compound represented by Chemical Formula 2 below (hereinafter “compound of Chemical Formula 2”):

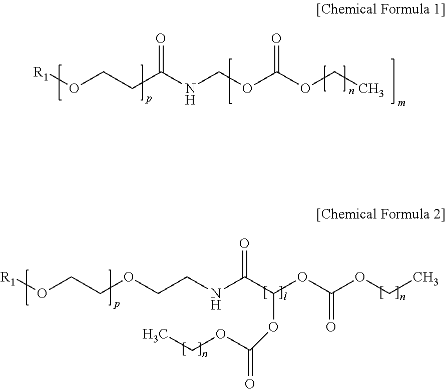

in Chemical Formula 1 or Chemical Formula 2,
R₁ represents hydrogen, a C₁ to C₅ alkyl group, an amine group, a C₁ to C₅ alkylamine group, a carboxyl group, or a C₁ to C₅ alkylcarboxyl group,
p is an integer of 12 to 227,
n is an integer of 2 to 14,
m is an integer of 1 to 3, and
l is an integer of 4.

In the present invention, the term “micelle” refers to a compound with a spherical structure formed by amphiphilic low molecular weight substances having both hydrophilic and hydrophobic properties. The micelle is thermodynamically stable. When a water-insoluble (hydrophobic) drug is dissolved in a compound having such micelle structure, the drug is present in the micelle.

The micelle of the present invention includes a compound in which an alkyl chloroformate is conjugated with a hydroxyl group of polyethylene glycol to form a carbonate group (—O—(C═O)—O—). In the present invention, the compound can be expressed as a polyethylene glycol derivative.

Specifically, the micelle has a carbonate bond between an alkyl chloroformate present in a hydrophobic core of the micelle and hydrophilic polyethylene glycol on a surface (or shell) of the micelle. Therefore, the alkyl chloroformate of the polyethylene glycol derivative is present in the micelle and the polyethylene glycol is present on the surface of the micelle.

The polyethylene glycol derivative constituting the carbon dioxide-generating micelle according to the present invention includes two or more carbonate groups in its structure, and under water-soluble conditions, the carbonate group of the micelle may be broken by hydrolysis, causing a reaction to generate carbon dioxide. Therefore, the micelle may generate carbon dioxide.

In a specific example, in the compound of Chemical Formula 1, q may be an integer of 4 to 10, an integer of 4 to 8, an integer of 6 to 10, or an integer of 8 to 10, n may be an integer of 3 to 10, and m may be an integer of 2 to 3 or an integer of 3.

In a specific example, the compound of Chemical Formula 1 may include a plurality of alkyl chloroformate moieties, specifically three, which makes it possible to generate much more carbon dioxide compared to an existing micelle. An amount of carbon dioxide that is generated may be increased in proportion to the number of carbonate groups, so a fat cell death rate may be improved.

In the present invention, depending on the number of m, for example, when m is 3, the compound of Chemical Formula 1 may be expressed as a tri-arm polyethylene glycol derivative.

In a specific example, in the compound of Chemical Formula 2, q may be an integer of 4 to 10, an integer of 4 to 8, an integer of 6 to 10, or an integer of 8 to 10, n may be an integer of 3 to 10, and I may be an integer of 4.

In a specific example, the compound of Chemical Formula 2 may include five alkyl chloroformate moieties. Therefore, the micelle of the present invention may generate much more carbon dioxide compared to an existing micelle.

In the present invention, depending on the number of 1, for example, when 1 is 4, the compound of Chemical Formula 2 may be expressed as a penta-arm polyethylene glycol derivative.

In a specific example, the compound of Chemical Formula 1 or the compound of Chemical Formula 2 may be prepared from polyethylene glycol having a molecular weight of 2,000 to 7,000 Da (g/mol) or 3,000 to 7,000 Da.

In a specific example, the micelle may have a diameter of 150 to 500 nm or 200 to 400 nm. When the diameter is too small, a desired fat cell killing effect is not achieved, and when the diameter is too large, it is inappropriate for injecting the micelle into the body, so it is recommended to adjust the diameter in the above-described range.

In a specific example, the compound of Chemical Formula 1 may be a compound represented by Chemical Formula 3 below.

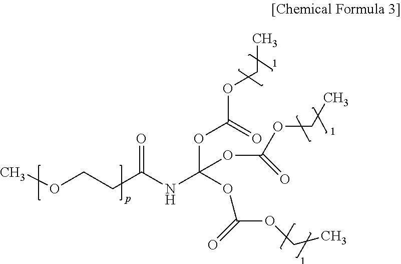

In a specific example, the compound of Chemical Formula 2 may be a compound represented by Chemical Formula 4 below.

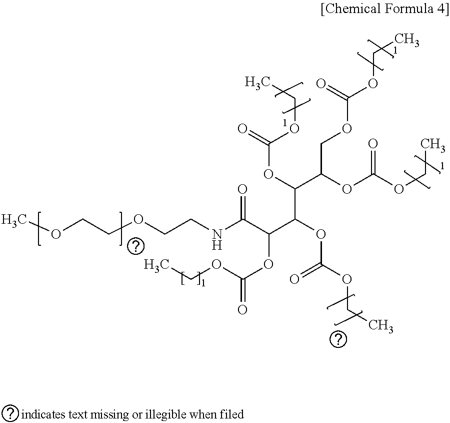

The carbon dioxide-generating micelle according to the present invention may destroy fat cells through carbon dioxide generated by hydrolysis.

The carbon dioxide-generating micelle according to the present invention may further include a compound represented by Chemical Formula 5 (hereinafter, “compound of Chemical Formula 5”) below along with one or more compounds selected from the group consisting of the compound of Chemical Formula 1 and the compound of Chemical Formula 2 described above.

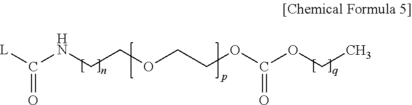

In Chemical Formula 5,
p is an integer of 12 to 227,
q is an integer of 2 to 14,
n is an integer of 0 to 5, and
L is a nona-arginine (r9) peptide.

In a specific example, q of the compound of Chemical Formula 5 may be an integer of 4 to 10, an integer of 4 to 8, an integer of 6 to 10, or an integer of 8 to 10, and n may be an integer of 1 to 3.

In a specific example, the compound of Chemical Formula 5 may have a structure in which a peptide is bonded to a surface of the micelle. The peptide may form a strong bond by binding to an end of polyethylene glycol on the surface of the micelle, and for example, a carboxyl group of the peptide and an amine group at an end of polyethylene glycol may be bonded. Due to the peptide, the micelle of the present invention may have a targeting ability.

In a specific example, the peptide may be a nona-arginine (r9) peptide, and the cell permeability of the micelle may be improved due to the nona-arginine (r9) peptide.

In a specific example, the compound of Chemical Formula 5 may be a compound represented by Chemical Formula 6 below.

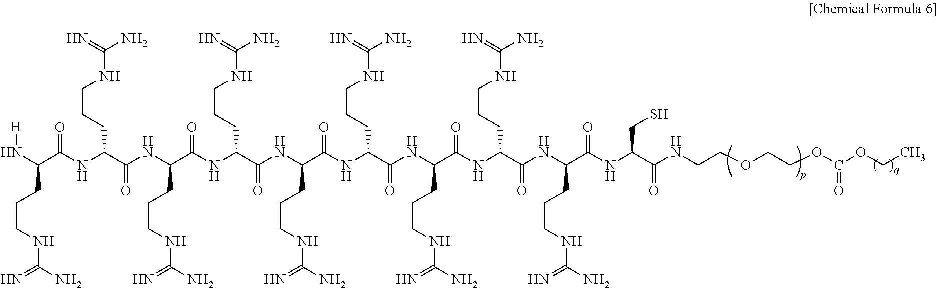

In Chemical Formula 6, p and q may be the same as p and q of the compound of Chemical Formula 5.

In specific example, a weight ratio of one or more compounds selected from the group consisting of the compound of Chemical Formula 1 and the compound of Chemical Formula 2, and the compound of Chemical Formula 5 may be 99:1 to 96:4.

In addition, the present invention relates to a method of preparing the carbon dioxide-generating micelle described above.

The carbon dioxide-generating micelle according to the present invention may be prepared using a method including: synthesizing a hydroxylated polyethylene glycol derivative, which is one or more compounds selected from the group consisting of a compound represented by Chemical Formula 1 below and a compound represented by Chemical Formula 2 below, by mixing hydroxylated polyethylene glycol and an alkyl chloroformate (step 1); and
synthesizing a micelle by dissolving the hydroxylated polyethylene glycol derivative in one or a mixed solvent of two or more selected from acetonitrile, methylene chloride, chloroform, and methanol and evaporating the solvent (step 2).

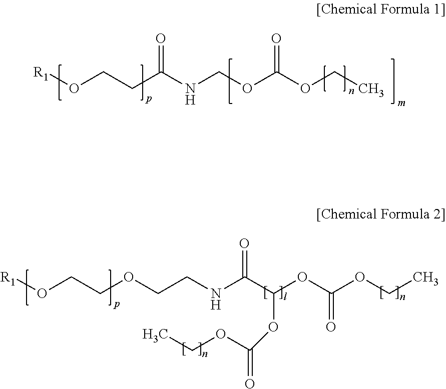

In the above Chemical Formula 1 or Chemical Formula 2,
R₁ represents hydrogen, a C₁ to C₅ alkyl group, an amine group, a C₁ to C₅ alkylamine group, a carboxyl group, or a C₁ to C₅ alkylcarboxyl group,
p is an integer of 12 to 227,
n is an integer of 2 to 14,
m is an integer of 1 to 3, and
l is an integer of 4.

Hereinafter, the method of preparing a carbon dioxide-generating micelle of the present invention will be described in further detail.

### Step 1. Synthesis of Hydroxylated Polyethylene Glycol Derivative

Before synthesizing a polyethylene glycol derivative, hydroxylated polyethylene glycol may be prepared.

The hydroxylated polyethylene glycol may be prepared by reacting polyethylene glycol with a compound with a hydroxyl group. The reaction may be a CDI reaction or EDC/NHS reaction.

In a specific example, polyethylene glycol may have a molecular weight of 2,000 to 7,000 Da (g/mol) or 3,000 to 7,000 Da but is not limited thereto. When the molecular weight is outside the above range, bubbles may be generated in a short period of time because the micelle itself is unstable. In particular, when the molecular weight is less than 2,000 Da, the micelle may not be formed.

In a specific example, polyethylene glycol with three hydroxyl groups may be prepared according to Reaction Scheme 1 below.

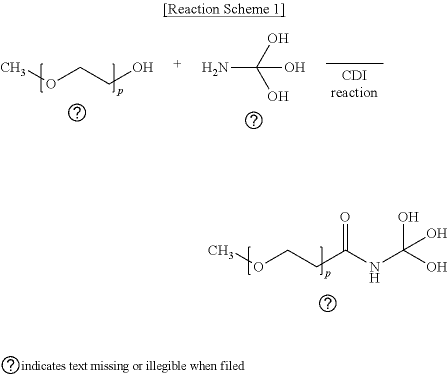

In a specific example, polyethylene glycol with five hydroxyl groups may be prepared according to Reaction Scheme 2 below.

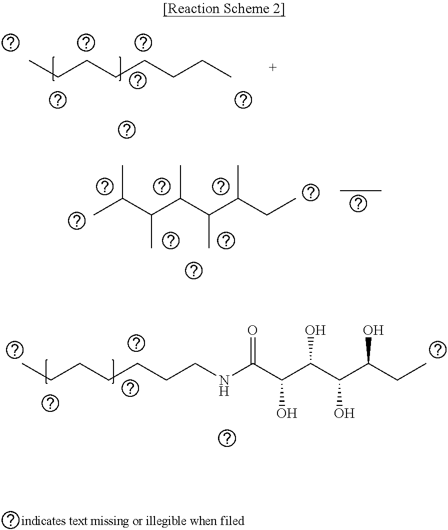

In the present invention, a hydroxylated polyethylene glycol derivative may be prepared by mixing hydroxylated polyethylene glycol and an alkyl chloroformate.

First, hydroxylated polyethylene glycol and an alkyl chloroformate are each dissolved in acetonitrile to prepare a hydroxylated polyethylene glycol solution and an alkyl chloroformate solution. The alkyl chloroformate solution is added to the hydroxylated polyethylene glycol solution, and the mixture is stirred. After stirring the mixture, pyridine is added to the stirred mixture and reacted to prepare hydroxylated polyethylene glycol-alkyl carbonate.

In a specific example, the alkyl chloroformate may be an aliphatic compound and be a chloroformate with an alkyl group having 4 to 10 carbon atoms, 4 to 8 carbon atoms, 6 to 10 carbon atoms, or 8 to 10 carbon atoms. For example, butyl chloroformate, octyl chloroformate, or dodecyl chloroformate may be used as the alkyl chloroformate but is not limited thereto.

In a specific example, the hydroxylated polyethylene glycol solution may be prepared by dissolving 0.05 to 0.8 mmol of hydroxylated polyethylene glycol in 2 to 6 ml of acetonitrile, and the alkyl chloroformate solution may be prepared by dissolving 1 to 3 mmol of alkyl chloroformate in 3 to 7 ml of acetonitrile.

In a specific example, the alkyl chloroformate solution may be added to the hydroxylated polyethylene glycol solution, and the mixed solution may be stirred for 2 to 10 minutes, 3 to 8 minutes, 4 to 6 minutes, or 5 minutes.

In a specific example, nitrogen may be flowed during the stirring process. Since reactants are sensitive to moisture in the air, the reaction in the present invention may be induced to occur stably by flowing nitrogen with low reactivity.

In a specific example, the hydroxylated polyethylene glycol derivative may be prepared by adding 0.5 to 3.5 mmol of pyridine to the stirred mixture, reacting the mixture at 0 to 5° C. for 20 to 40 minutes, and then reacting the mixture at room temperature for 24 hours.

The hydroxylated polyethylene glycol derivative is hydroxylated polyethylene glycol-alkyl carbonate.

In a specific example, the compound of Chemical Formula 3, which is a hydroxylated polyethylene glycol derivative, may be prepared according to Reaction Scheme 3 below.

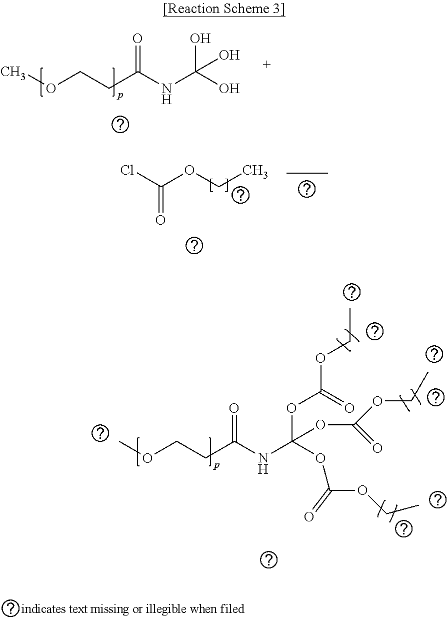

In addition, in a specific example, the compound of Chemical Formula 4, which is a hydroxylated polyethylene glycol derivative, may be prepared according to Reaction Scheme 4 below.

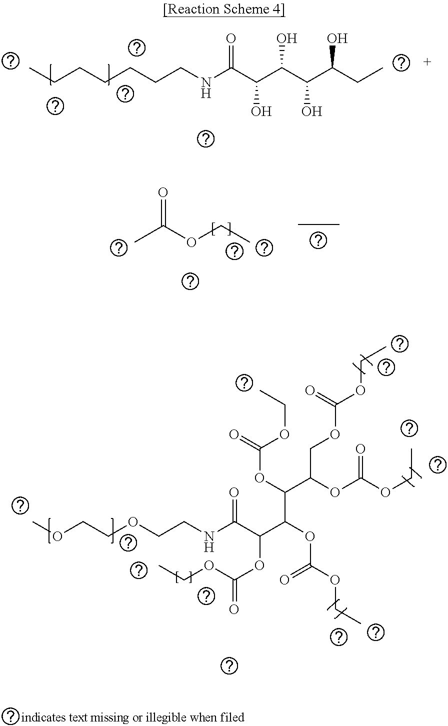

### Step 2. Preparation of Micelle Particles Using Solvent Evaporation

A micelle may be prepared from the hydroxylated polyethylene glycol derivative prepared in step 1, using solvent evaporation.

Specifically, the micelle may be prepared by dissolving the hydroxylated polyethylene glycol derivative in an organic solvent, performing a solvent evaporation method to volatilize the solvent, and then redispersing the resulting mixture in a hydrophilic solution.

In a specific example, 5 to 15 mg of the hydroxylated polyethylene glycol derivative may be dissolved in the organic solvent. The organic solvent may be a commonly used organic solvent such as acetonitrile, methylene chloride, chloroform, and methanol.

In addition, the organic solvent may be a mixed solvent of methylene chloride and acetonitrile, a mixed solvent of methylene chloride and chloroform, and a mixed solvent of methylene chloride and methanol.

In the mixed solvent used in the present invention, a ratio of methylene chloride and other solvents may be 3 to 1:1 to 3.

In a specific example, the hydroxylated polyethylene glycol derivative may be dissolved in the organic solvent and then applied to a glass wall using a concentrator under vacuum at 100 to 300 rpm, 150 to 200 rpm, or 180 rpm, at 25 to 45° C., 30 to 40° C., or 37° C. for about 5 to 10 minutes. In the present invention, the glass wall may be uniformly coated because a concentrator is used to coat the glass wall. In the case of a solvent evaporation method using nitrogen, additional efforts are required to adjust rotation speed and rotation angle constantly because the solvent is evaporated manually, but in the present invention, as a concentrator is used to coat the glass wall, continuous and uniform coating is easily achievable.

In addition, in a specific example, the hydrophilic solution may include PBS and distilled water.

In the present invention, when preparing micelle particles using a solvent evaporation method, the micelle may be prepared using the hydroxylated group polyethylene glycol derivative and the compound of Chemical Formula 5.

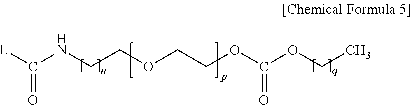

In Chemical Formula 5,
p is an integer of 12 to 227,
q is an integer of 2 to 14,
n is an integer of 0 to 5, and
L is a nona-arginine (r9) peptide.

In other words, in step 2 of the present invention, the micelle may be synthesized by dissolving the hydroxylated polyethylene glycol derivative and the compound represented by Chemical Formula 5 in one or a mixed solvent of two or more selected from acetonitrile, methylene chloride, chloroform, and methanol and evaporating the solvent.

In the present invention, the compound of Chemical Formula 5 may be prepared by synthesizing the polyethylene glycol derivative by mixing polyethylene glycol and an alkyl chloroformate (hereinafter, “step a”); and
synthesizing a peptide-conjugated polyethylene glycol derivative by conjugating a nona-arginine (r9) peptide to the polyethylene glycol derivative (hereinafter, “step b”).

### Step a. Preparation of Polyethylene Glycol Derivative

In a specific example, step a may be performed in the same manner as step 1, except that polyethylene glycol is used instead of hydroxylated polyethylene glycol.

### Step b. Synthesis of Peptide-Conjugated Polyethylene Glycol Derivative

By conjugating a peptide to the polyethylene glycol derivative prepared in step a, a fat cell-targeting ability may be improved.

A peptide-conjugated polyethylene glycol is prepared through the EDC/NHS reaction of a compound having an amino group and a peptide.

In a specific example, the compound having an amino group may be aminopolyethylene glycol, and the peptide may be a nona-arginine (r9) peptide.

In a specific example, a molar ratio of the compound having an amino group and the peptide may be 1:0.1 to 1:10, or 1:0.5 to 1:3, and the peptide-conjugated polyethylene glycol derivative may be prepared through the EDC/NHS reaction.

In a specific example, the prepared peptide-conjugated polyethylene glycol may be dialyzed, and impurities and unreacted substances may be removed through a filter.

### Step c. Preparation of Micelle Particles by Solvent Evaporation Method Using Hydroxylated Polyethylene Glycol Derivative and Peptide-Conjugated Polyethylene Glycol Derivative

A micelle may be prepared from the hydroxylated polyethylene glycol derivative prepared in step 1 and the peptide-conjugated polyethylene glycol derivative prepared in step b using a solvent evaporation method.

Specifically, the micelle may be prepared by dissolving the hydroxylated polyethylene glycol derivative and peptide-conjugated polyethylene glycol derivative in an organic solvent, evaporating the solvent using a solvent evaporation method, and redispersing the resulting mixture in a hydrophilic solution.

In a specific example, a molar ratio of one or more compounds selected from the group consisting of the compound of Chemical Formula 1 and the compound of Chemical Formula 2, and the compound of Chemical Formula 3 may be 99:1 to 96:4.

In a specific example, 5 to 15 mg of the hydroxylated polyethylene glycol derivative and the peptide-conjugated polyethylene glycol derivative may be dissolved in the organic solvent. The organic solvent may be a commonly used organic solvent such as acetonitrile, methylene chloride, chloroform, and methanol.

The organic solvent may be a mixed solvent of methylene chloride and acetonitrile, a mixed solvent of methylene chloride and chloroform, and a mixed solvent of methylene chloride and methanol.

In the mixed solvent used in the present invention, a ratio of methylene chloride and other solvents may be 3 to 1:1 to 3.

In a specific example, the hydroxylated polyethylene glycol derivative and the peptide-conjugated polyethylene glycol derivative may be dissolved in the organic solvent and then applied to a glass wall using a concentrator under vacuum at 100 to 300 rpm, 150 to 200 rpm, or 180 rpm, at 25 to 45° C., 30 to 40° C., or 37° C. for about 5 to 10 minutes. In the present invention, the glass wall may be coated uniformly because a concentrator is used to coat the glass wall. In the case of a solvent evaporation method using nitrogen, additional efforts are required to adjust rotation speed and rotation angle constantly because the solvent is evaporated manually, but in the present invention, as a concentrator is used to coat the glass wall, continuous and uniform coating is easily achievable.

In a specific example, the hydrophilic solution may include PBS and distilled water.

Additionally, the present invention relates to a composition for fat reduction, which includes the above-described carbon dioxide-generating micelle.

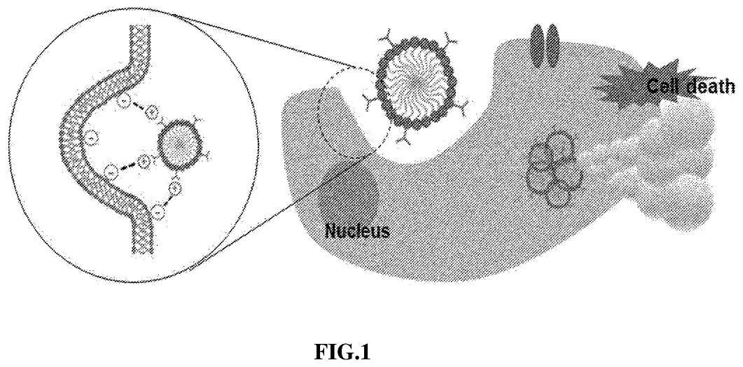

The carbon dioxide-generating micelle of the present invention includes a carbonate group in its structure, and under water-soluble conditions, the carbonate group of the micelle is broken by hydrolysis, causing a reaction to generate carbon dioxide. Therefore, the carbon dioxide-generating micelle of the present invention is locally injected in the form of nanoparticles and deposited in fat cells to generate gas. As the generated carbon dioxide damages fat cells, necrosis of fat cells occurs, which may reduce fat (FIG. 1).

The damage to fat cells may occur after the micelle is injected into the cells by controlling a structure of the carbon dioxide-generating micelle and controlling a carbon dioxide generation amount and time.

The composition for fat reduction according to the present invention may include the micelle including one or more compounds selected from the compound of Chemical Formula 1 and the compound of Chemical Formula 2.

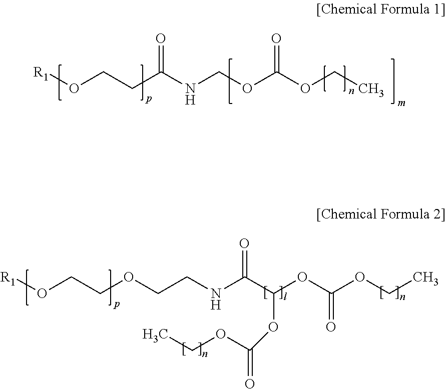

In Chemical Formula 1 or Chemical Formula 2,
R₁ represents hydrogen, a C₁ to C₅ alkyl group, an amine group, a C₁ to C₅ alkylamine group, a carboxyl group, or a C₁ to C₅ alkylcarboxyl group,
p is an integer of 12 to 227,
n is an integer of 2 to 14,
m is an integer of 1 to 3, and
l is an integer of 4.

The composition for fat reduction according to the present invention may include the micelle including one or more compounds selected from the group consisting of the compound of Chemical Formula 1 and the compound of Chemical Formula 2, and the compound of Chemical Formula 5 below.

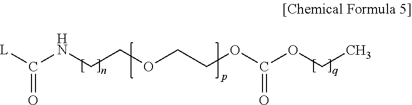

In Chemical Formula 5,
p is an integer of 12 of 227,
q is an integer of 2 to 14,
n is an integer of 0 to 5, and
L is a nona-arginine (r9) peptide.

In a specific example, a ratio of one or more compounds selected from the group consisting of the compound of Chemical Formula 1 and the compound of Chemical Formula 2, and the compound of Chemical Formula 5 may be 99:1 to 96:4.

In a specific example, the content of carbon dioxide-generating micelles in the composition for fat reduction may vary depending on the application area and the like, and may be, for example, 0.01 to 1.0 parts by weight or 0.1 to 0.5 parts by weight based on a total weight of the composition.

In a specific example, the composition for fat reduction according to the present invention may be used for local or intravenous injection and applied to body parts such as the chin, thighs, arms, and belly, which are frequently treated.

In a specific example, the composition for fat reduction according to the present invention may be used as a lipolytic supplement for local fat decomposition, a formulation corrector, or a cosmetic product for weight loss.

Hereinafter, examples of the present invention will be described in more detail. These examples are described solely to explain the present invention in more detail, and it is obvious to those skilled in the art that the scope of the present invention is not limited to these examples of the present invention.

### MODES OF THE INVENTION

### <Reference> Experimental Materials

Polyethylene glycol was purchased from Sigma-Aldrich. A number average molecular weight (Mn) of polyethylene glycol that is usable is 550 to 20,000, and polyethylene glycol having a number average molecular weight of 2,000 or 5,000 is preferable for preparing the carbon dioxide-generating micelle and was used in this experiment.

Octyl chloroformate (from Sigma-Aldrich), which is an aliphatic compound, was used as the alkyl chloroformate.

### EXAMPLES

### Preparation Example 1-1. Synthesis of Polyethylene Glycol with Three Hydroxyl Groups

By introducing three hydroxyl groups into polyethylene glycol, mPEG-tri arm (PEG-T) was prepared.

Specifically, 50 mM of polyethylene glycol was dissolved in dioxane, and 0.5 M of carbonylimidazole was added. After reacting the mixture at 37° C. for 2 hours, unreacted substances and impurities were separated using a 1000 MWCO dialysis membrane. After separation, a freeze-dried imidazole carbamate intermediate was produced.

The intermediate and tris(hydroxymethyl)aminomethane were added to a sodium carbonate buffer with a pH of 9 to 10, and unreacted substances and impurities were separated using a 1000 MWCO dialysis membrane.

Then, as shown in Reaction Scheme 1 below, hydroxyl groups are introduced into polyethylene glycol.

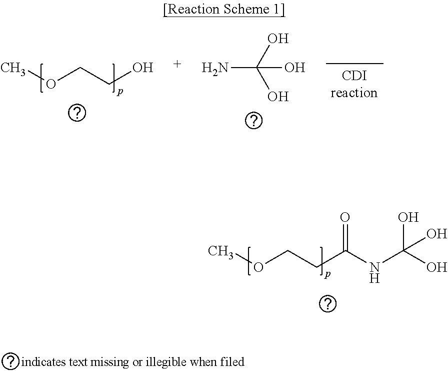

The prepared polyethylene glycol with three hydroxyl groups may be expressed as PEG_{mw}-tri arm (PEG_{mw}-T).

### Preparation Example 1-2. Synthesis of Polyethylene Glycol Derivative with Three Hydroxyl Groups

PEG_{mw}-T prepared in Preparation Example 1 and an alkyl chloroformate were each dissolved in acetonitrile. Specifically, 0.5 mmol of PEG_{mw}-T was dissolved in 4 ml of acetonitrile to prepare a polyethylene glycol solution, and 2 mmol of the alkyl chloroformate was added to 5 ml of acetonitrile to prepare an alkyl chloroformate solution.

The alkyl chloroformate solution was added to the polyethylene glycol solution, and the mixture was stirred for 5 minutes. Nitrogen with low reactivity was flowed during the stirring process. 2.5 mmol of pyridine was added to the stirred mixture and reacted at 0° C. for 30 minutes. After the reaction was completed, the mixture was stirred at room temperature for 24 hours to complete the synthesis.

The synthesized solution was precipitated in diethyl ether, filtered through a filter, and dried in a vacuum dryer for 3 to 7 days to obtain a polyethylene glycol derivative, which is a synthetic polymer, that is, polyethylene glycol-alkyl carbonate.

Different types of polyethylene glycol derivatives, that is, polyethylene glycol-alkyl carbonate, may be produced depending on the number average molecular weight of polyethylene glycol and the type of alkyl of the alkyl chloroformate. For example, when the number average molecular weight of polyethylene glycol is 5000 and octyl chloroformate is used, the polyethylene glycol may be expressed as PEG₅₀₀₀-octylcarbonate or PEG₅₀₀₀-OC.

The prepared polyethylene glycol derivative with three hydroxyl groups may be expressed as PEG_{mw}-tri arm-octylcarbonate (PEG_{mw}-T-OC).

### Preparation Example 2-1. Synthesis of Polyethylene Glycol with Five Hydroxyl Groups

Sodium gluconate was dissolved in a 0.1M MES/0.3M NaCl buffer. After 10 minutes, 1-ethyl-3-(3-dimethylaminopropyl)carbodiimide was added, and after another 10 minutes, sulfo-N-hydroxysuccinimide was added, and after another 10 minutes, methoxypolyethylene glycolamine was added and reacted for one day to synthesize polyethylene glycol with five hydroxyl groups through the EDC/NHS reaction.

Then, unreacted substances and impurities were separated through a 1000 MWCO dialysis membrane for 3 days, and freeze-drying was performed.

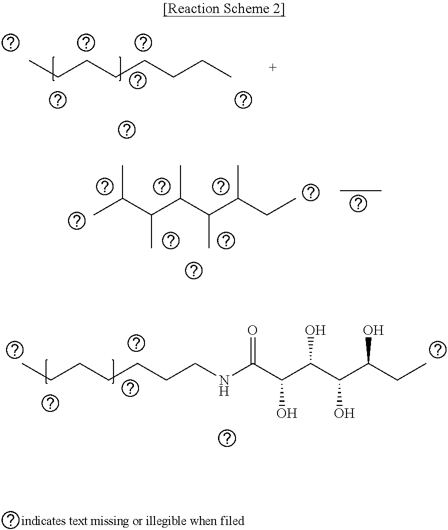

The prepared polyethylene glycol with five hydroxyl groups may be expressed as PEGmw-penta arm (PEGmw-P).

### Preparation Example 2-2. Synthesis of Polyethylene Glycol Derivative with Five Hydroxyl Groups

0.1 mmol of PEGmw-P prepared in Preparation Example 2-1 was dissolved in 4 ml of acetonitrile to prepare a polyethylene glycol solution. An alkyl chloroformate solution was prepared by dissolving 2 mmol of alkyl chloroformate in 5 ml of acetonitrile.

Nitrogen with low reactivity was flowed into the polyethylene glycol solution, and after 20 minutes, the alkyl chloroformate solution was added and stirred for 5 minutes. After the stirring process, 1 mmol of pyridine was added to the stirred mixture and reacted at 0° C. for 30 minutes. The mixture was stirred at room temperature for 24 hours to complete the reaction.

After the reaction was completed, the solution was precipitated in diethyl ether, and the supernatant, excluding the precipitate, was removed through centrifugation. After repeating the process 5 times, the precipitate was dried in a freeze dryer for 3 days to obtain a polyethylene glycol derivative, which is a synthetic polymer, that is, polyethylene glycol-alkyl carbonate.

The prepared polyethylene glycol derivative with five hydroxyl groups may be expressed as PEG_{mw}-penta arm-octylcarbonate (PEG_{mw}-P-OC).

### Preparation Example 3. Synthesis of Polyethylene Glycol Derivative

A polyethylene glycol derivative was prepared in the same manner as Preparation Example 1-2, except that polyethylene glycol was used instead of PEG_{mw}-T.

The prepared polyethylene glycol derivative may be expressed as PEG_{mw}-octylcarbonate (PEG_{mw}-OC).

### Preparation Example 4. Synthesis of Peptide-Conjugated Polyethylene Glycol Derivative

The introduction of a peptide into the polyethylene glycol derivative prepared in Preparation Example 3 was performed as follows.

A peptide-conjugated polyethylene glycol derivative was prepared by subjecting an aminopolyethylene glycol derivative having an amino group and a peptide at a molar ratio of 1:1 to an EDC/NHS reaction. Then, the peptide-conjugated polyethylene glycol derivative was dialyzed for 4 days, and impurities and unreacted substances were removed through a filter, and freeze-drying was performed.

The prepared peptide-conjugated polyethylene glycol derivative may be expressed as r9-PEG_{mw}-octylcarbonate (r9-PEG_{mw}-OC).

### Preparation Example 5. Preparation of Micelle Particles Using Solvent Evaporation

A micelle was prepared by a solvent evaporation method using a mixed solvent including methylene chloride and acetonitrile in a ratio of 2:1.

The hydroxylated polyethylene glycol derivative prepared in Preparation Example 1-2 or Preparation Example 2-2 and/or the peptide-conjugated polyethylene glycol derivative prepared in Preparation Example 4 was dissolved in the mixed solvent and applied to a glass wall using a concentrator (N-1300) under vacuum at 180 rpm, at 37° C. for about 5 to 10 minutes, and as a result, a surface of the glass was coated with particles of the derivative (solvent evaporation).

Self-assembly was performed by adding a hydrophilic solution (including PBS and distilled water) after evaporating the solvent, and the micelle was prepared.

In Preparation Examples, depending on derivatives used to prepare the micelle, micelles are expressed as follows.
PEG_{mw}-OC micelle: Preparation of a micelle using PEG_{mw}-OC
PEG_{mw}-T-OC micelle: Preparation of a micelle using PEG_{mw}-T-OC
PEG_{mw}-P-OC micelle: Preparation of a micelle using PEG_{mw}-P-OC
r9-PEG_{mw}-OC micelle: Preparation of a micelle using PEG_{mw}-OC and r9-PEG_{mw}-OC
r9-PEG_{mw}-T-OC micelle: Preparation of a micelle using PEG_{mw}-T-OC and r9-PEG_{mw}-OC

In r9-PEG_{mw}-OC micelles used in Experimental Examples 5 to 7 below, a mixing ratio (weight ratio) of PEG_{mw}-OC and r9-PEG_{mw}-OC is 50:1.

In r9-PEG_{mw}-T-OC micelles used in Experimental Examples 5 to 7, described below, a mixing ratio (weight ratio) of PEG_{mw}-T-OC and r9-PEG_{mw}-OC is 50:1.

### Experimental Example 1. Confirmation of Synthesis of Hydroxylated Polyethylene Glycol (PEG-T)

The synthesis of polyethylene glycol with three hydroxyl groups (PEG_{mw}-T) prepared in Preparation Example 1-1 was confirmed. To confirm the synthesis, a molecular weight was measured using nuclear magnetic resonance analysis.

TABLE 1

| | | |
|---|---|---|
| Molecular weight of PEG (mw) | 2,000 | 5,000 |
| PEG_{mw} | 0.216 | 0.152 |
| PEG_{mw}-tri arm | 0.512 | 0.288 |

In the present invention, Table 1 illustrates an increase in the number of hydroxyl groups according to a molecular weight of polyethylene glycol.

As shown in Table 1, as a result of measuring the molecular weight of hydroxylated polyethylene glycol, it can be seen that the molecular weight increased by 2.3 times when polyethylene glycol with a molecular weight of 2,000 was used while the molecular weight increased by 1.9 times when polyethylene glycol with a molecular weight of 5,000 was used. From this result, polyethylene glycol in which a hydroxyl group was introduced was confirmed.

### Experimental Example 2. Confirmation of Gas Generation Time of Hydroxylated Polyethylene Glycol Derivatives (PEG-T Derivative)

Experimental Example 2 was conducted to confirm the gas generation time of the hydroxylated polyethylene glycol derivative prepared in Preparation Example 1-2. The gas generation was confirmed using an ultrasonic device (SONON 300L). mPEG₂₀₀₀-triarm-octylcarbonate (PEG₂₀₀₀-T-OC) and mPEG₅₀₀₀-triarm-octylcarbonate (PEG₅₀₀₀-T-OC), which are PEG_{mw}-T derivatives according to the present invention, were used as experimental groups, and mPEG₂₀₀₀-octylcarbonate (PEG₂₀₀₀-OC) and mPEG₅₀₀₀-octylcarbonate (PEG₅₀₀₀-OC), which are polyethylene glycol derivatives of Preparation Example 3, were used as control groups.

Specifically, the CO₂ gas generation rate of the experimental and control groups was confirmed.

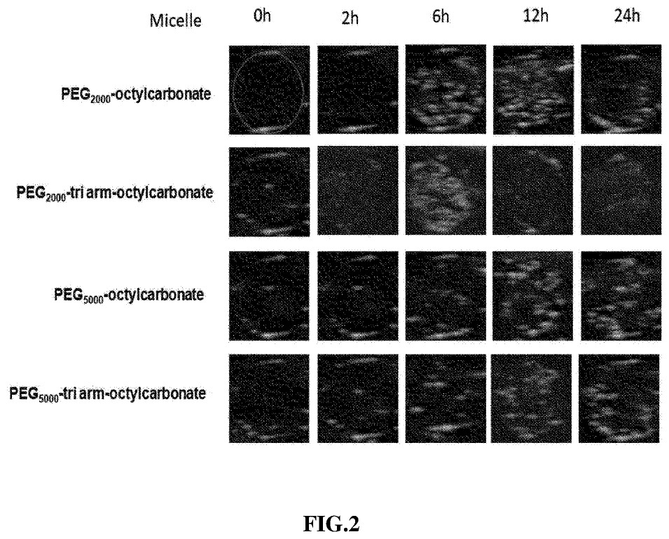

As a result of analysis, it can be seen that a carbon dioxide generation time varies depending on whether a hydroxyl group is introduced and a molecular weight. When the molecular weight of polyethylene glycol was 2,000, carbon dioxide was generated at a much faster rate. On the other hand, when the molecular weight of polyethylene glycol was 5,000, there was no significant difference depending on whether a hydroxyl group was introduced (FIG. 2).

### Experimental Example 3. Analysis of Fat Cell Killing Ability of Peptide-Conjugated Micelles

In this experiment example, the fat cell killing effect was confirmed depending on an amount of a peptide (r9 peptide), which is bonded on a surface of the micelle for cell penetration.

In this experimental example, r9-PEG_{mw}-OC micelles prepared in Preparation Example 5 were used, and at this time, an amount of peptide-conjugated polyethylene glycol derivative was 0% to 4% of a total weight of the derivative (r9-PEG_{mw}-OC/PEG_{mw}-OC).

The fat cell killing ability was measured according to the MTS assay.

Specifically, 3T3-11 cells were seeded in 96-well plates at 2×10³ cells/plate, and then differentiation was performed for 2 or 3 weeks. The derivative was treated at a concentration of 0.2 wt % and 24 hours later, the cells was washed once with PBS. Absorbance was analyzed 1 hour after treatment with an MTS solution.

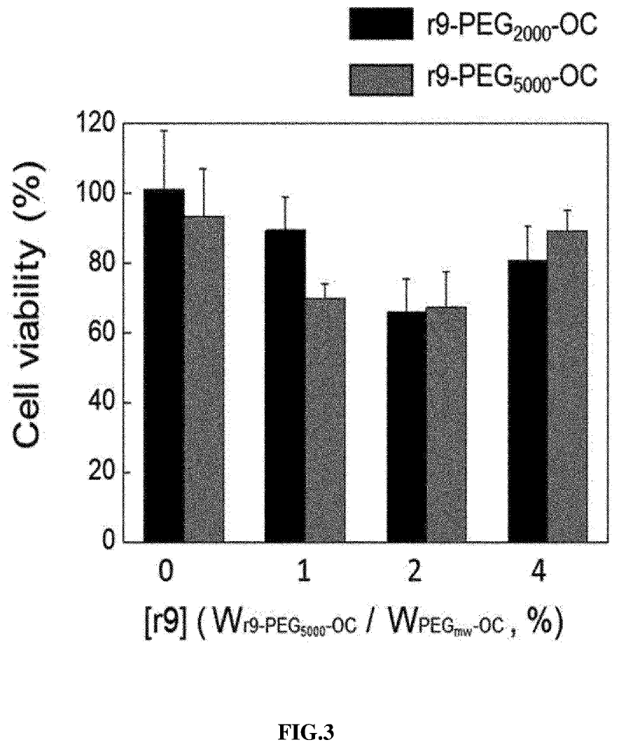

The results are shown in FIG. 3.

As shown in FIG. 3, it can be seen that the fat cell killing effect tends to occur depending on the amount of introduced peptide.

In preparing micelles for killing fat cells, an appropriate concentration of peptide/derivative varies depending on a molecular weight of polyethylene glycol, which is 2-4% when the derivative with a molecular weight of 2,000 is used and 1-4% when the derivative with a molecular weight of 5,000 is used.

### Experimental Example 4. Fat Cell Death in Gas Non-Generating Groups

In this experimental example, fat cell death in gas non-generating groups was analyzed.

The analysis was performed according to the method of Experimental Example 2, and PEG₅₀₀₀-octylamine micelles, r9-PEG₅₀₀₀-octylamine micelles, and r9-PEG₅₀₀₀-tri arm-octylamine micelles were used as gas non-generating micelles based on polyethylene glycol.

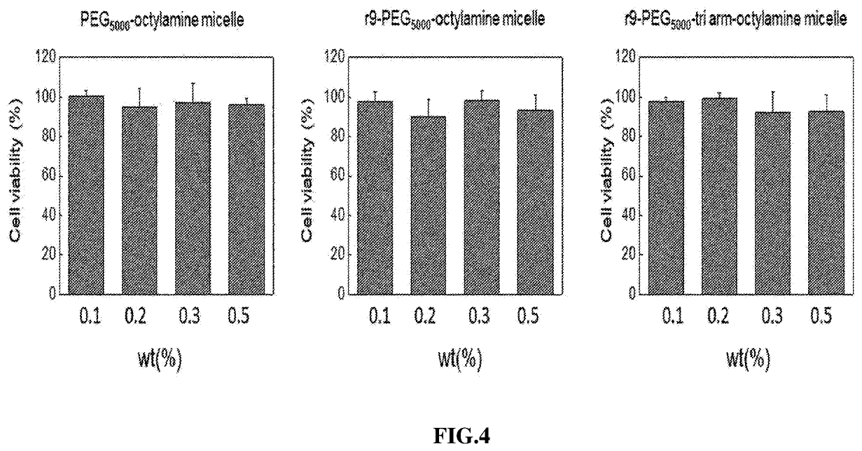

The results are shown in FIG. 4.

As shown in FIG. 4, fat cell death did not occur when gas was not generated. It can be seen that cell viability was at least 90% in all groups and at all concentrations.

### Experimental Example 5. Fat Cell Death According to Introduction of Peptide and Micelle Concentration

The micelles prepared in Preparation Example 5 were treated at a concentration of 0.1 to 0.5 wt % to evaluate fat cell killing ability.

Specifically, the fat cell killing ability of PEG₅₀₀₀-octylcarbonate micelles (PEG_{mw}-OC micelles), PEG₅₀₀₀-triarm-octylcarbonate micelles (PEG_{mw}-T-OC micelles), r9-PEG₅₀₀₀-octylcarbonate micelles (r9-PEG_{mw}-OC micelles), and r9-PEG₅₀₀₀-triarm-octylcarbonate micelles (r9-PEG_{mw}-T-OC micelles) was evaluated.

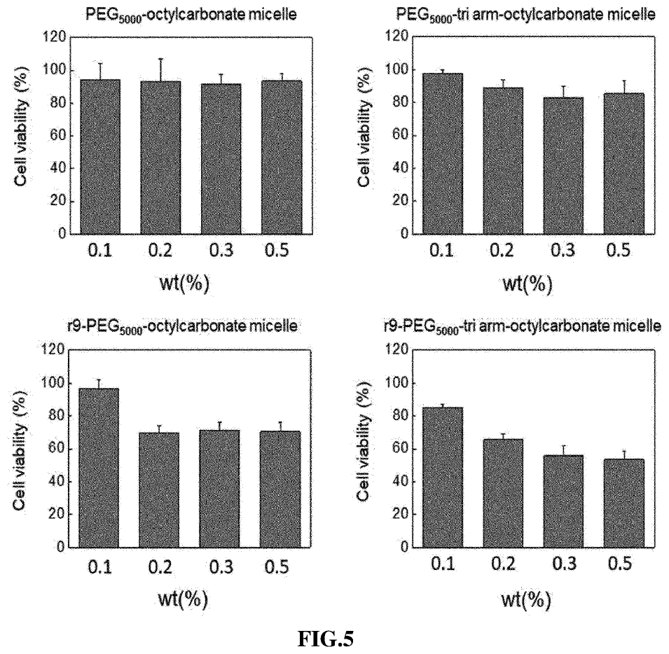

The results are shown in FIG. 5.

As shown in FIG. 5, as a result of confirming cell death in differentiated adipocytes according to the concentration of polyethylene-based carbon dioxide-generating micelles with a molecular weight of 5,000, it was found that the group, in which no peptide was introduced, showed almost no cell death and high cell viability. Specifically, cell viability of at least 83% was observed.

It can be seen that the greatest number of killed fat cells occurred in the case of the micelles, in which carbon dioxide generation was increased by introducing hydroxyl groups into polyethylene glycol. In addition, it can be seen that more fat cells were killed in the groups having hydroxyl groups compared to the groups having no hydroxyl groups.

### Experimental Example 6. Confirmation of In Vivo Efficacy

After injecting micelles into adipose tissue of obesity-induced mice, weight changes were observed and compared to adipose tissue in which micelles were not injected.

Obesity-induced mice were fed a high-fat diet for about 10 weeks before micelle injection. A single dose of micelles at a concentration of 25 mg/kg was injected into the left fat pad of an obesity-induced mouse weighing over 40 g, and after monitoring weight changes for 3 weeks, both the fat pad, where micelles were injected, and the opposite (non-injected) fat pad were extracted to confirm the treatment effect based on the weight ratio.

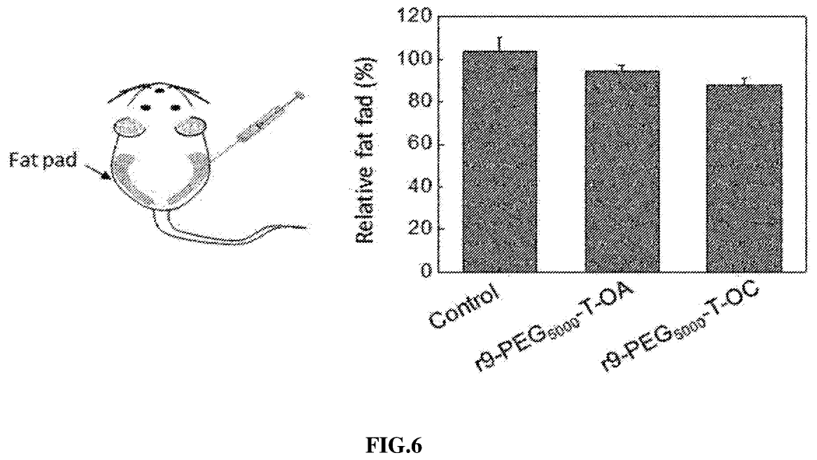

The results are shown in FIG. 6.

As shown in FIG. 6, it can be seen that the weight of the fat pad was relatively reduced in the carbon dioxide-generating micelles (r9-PEG₅₀₀₀-T-OC micelles; r9-PEG₅₀₀₀-tri arm-octylcarbonate micelles) according to the present invention compared to the gas non-generating group (r9-PEG₅₀₀₀-T-OA micelles; r9-PEG5000-tri arm-octylamine micelles).

In order to confirm the efficacy of the micelle as an obesity treatment, fat tissue was extracted, stained, and histologically analyzed. Hematocyline & Eosin (H&E) staining was used for histological analysis.

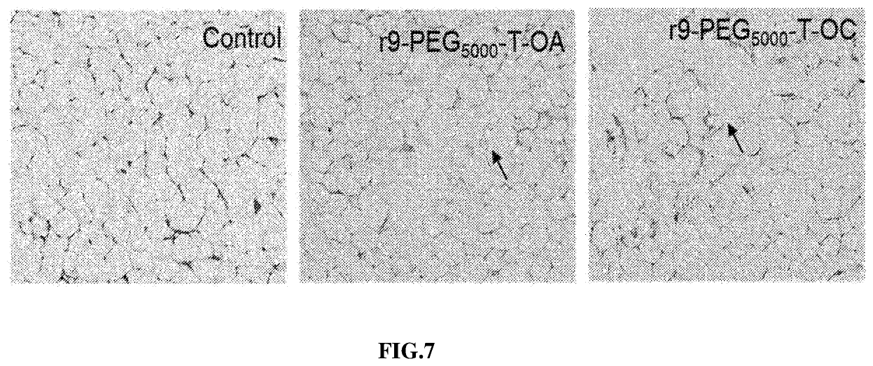

The results are shown in FIG. 7.

As shown in FIG. 7, compared to the gas non-generating group (r9-PEG₅₀₀₀-T-OA micelles), cell death and tissue reduction were observed in the carbon dioxide-generating micelles (r9-PEG₅₀₀₀-T-OC micelles) according to the present invention (black arrow: injection site).

### Experimental Example 7. Confirmation of Retention of Peptide-Conjugated Micelles

The retention of micelles in PEGmw-T-OC micelles and r9-PEGmw-T-OC micelles was confirmed by a biodistribution test using fluorescently labeled drugs.

Micelles were locally injected into the left fat pad in the same manner as Experimental Example 6 described above, and 24 hours or 48 hours after the injection, the retention of micelles was relatively compared by measuring fluorescence intensity.

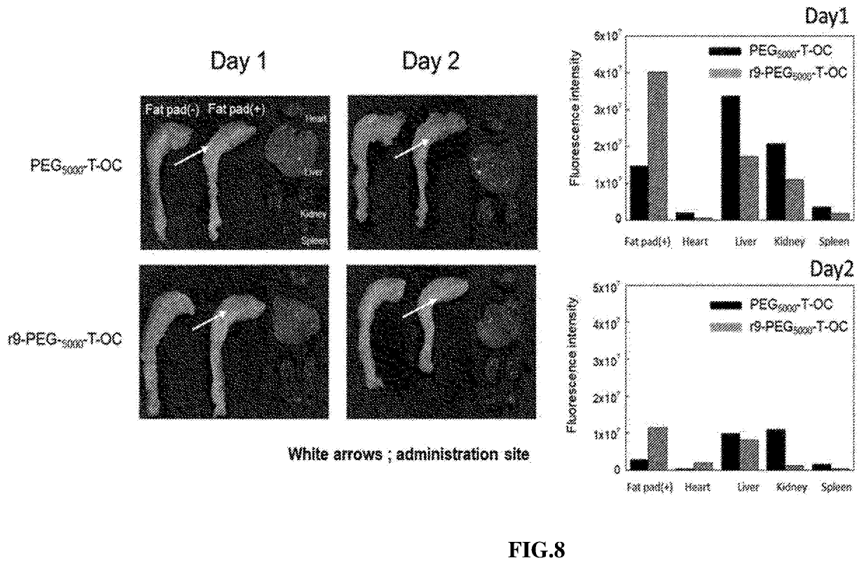

The results are shown in FIG. 8.

Through fluorescently labeled micelles, it can be seen that r9-PEG₅₀₀₀-T-OC micelles remained at the injection site for a longer period of time compared to PEG₅₀₀₀-T-OC micelles, in which no peptide was introduced.

The micelles may undergo hydrolysis continuously. The micelles without a peptide rapidly move to the liver and kidneys, which may be taken up by liver and kidney cells, generate gas, and even cause side effects. Therefore, such side effects may be minimized by introducing a peptide as in the present invention.

### INDUSTRIAL APPLICABILITY

The carbon dioxide-generating micelle according to the present invention can be produced as an injectable preparation and used for cosmetic treatment for weight loss and obesity treatment such as a lipolytic supplement to break down localized fat, a formulation corrector, or a cosmetic product for weight loss.

1. A carbon dioxide-generating micelle comprising one or more compounds selected from the group consisting of a compound represented by Chemical Formula 1 below and a compound represented by Chemical Formula 2 below: 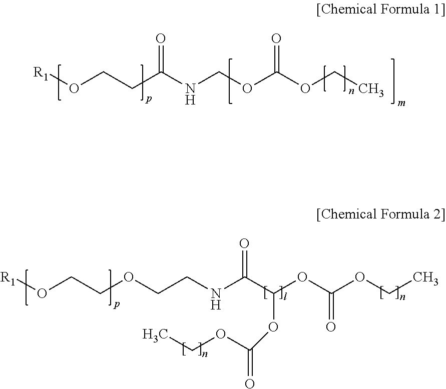
in Chemical Formula 1 or Chemical Formula 2,
R₁ represents hydrogen, a C₁ to C₅ alkyl group, an amine group, a C₁ to C₅ alkylamine group, a carboxyl group, or a C₁ to C₅ alkylcarboxyl group,
p is an integer of 12 to 227,
n is an integer of 2 to 14,
m is an integer of 1 to 3, and
l is an integer of 4.

2. The carbon dioxide-generating micelle of claim 1, wherein the micelle has a diameter of 150 to 500 nm.

3. The carbon dioxide-generating micelle of claim 1, wherein the compound represented by Chemical Formula 1 is a compound represented by Chemical Formula 3 below, and the compound represented by Chemical Formula 2 is a compound represented by Chemical Formula 4 below:

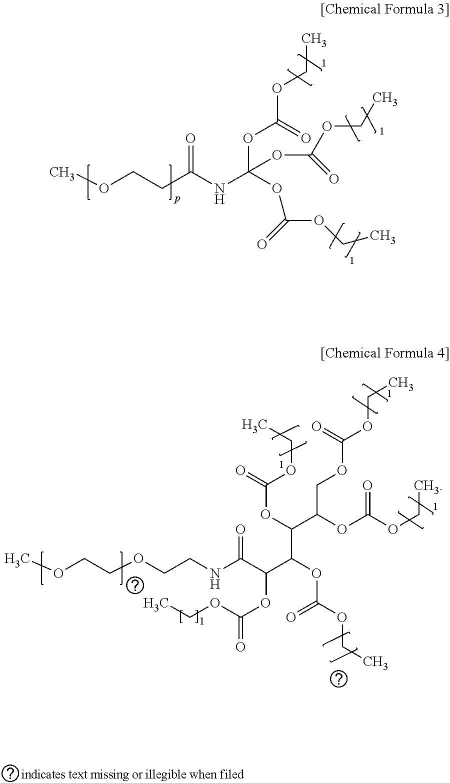

4. The carbon dioxide-generating micelle of claim 1, wherein a fat cell is destroyed by carbon dioxide generated by hydrolysis of one or more compounds selected from the group consisting of the compound represented by Chemical Formula 1 and the compound represented by Chemical Formula 2.

5. The carbon dioxide-generating micelle of claim 1, further comprising a compound represented by Chemical Formula 5 below: 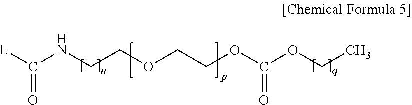
in Chemical Formula 5,
p is an integer of 12 to 227,
q is an integer of 2 to 14,
n is an integer of 0 to 5, and
L is a nona-arginine (r9) peptide.

6. The carbon dioxide-generating micelle of claim 5, wherein the compound represented by Chemical Formula 5 is a compound represented by Chemical Formula 6 below: 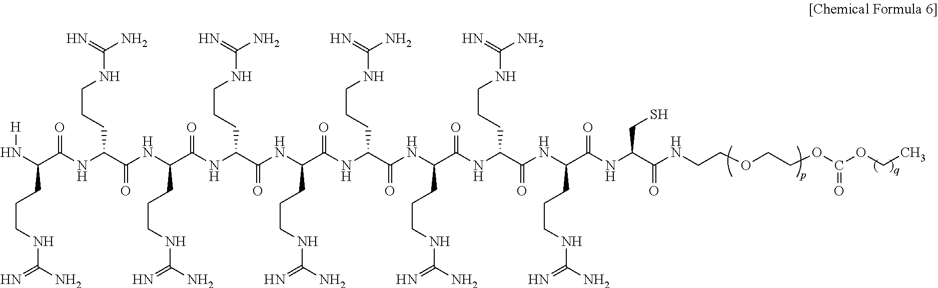
in Chemical Formula 6,
p is an integer of 12 to 227, and
q is an integer of 2 to 14.

7. The carbon dioxide-generating micelle of claim 5, wherein a weight ratio of one or more compounds selected from the group consisting of the compound represented by Chemical Formula 1 and the compound represented by Chemical Formula 2 and the compound represented by Chemical Formula 3 is 99:1 to 96:4.

8. A method of preparing a carbon dioxide-generating micelle, comprising:
synthesizing a hydroxylated polyethylene glycol derivative, which is one or more compounds selected from the group consisting of a compound represented by Chemical Formula 1 below and a compound represented by Chemical Formula 2 below, by mixing hydroxylated polyethylene glycol and an alkyl chloroformate (step 1); and
synthesizing a micelle by dissolving the hydroxylated polyethylene glycol derivative in one or a mixed solvent of two or more selected from acetonitrile, methylene chloride, chloroform, and methanol and evaporating the solvent (step 2): 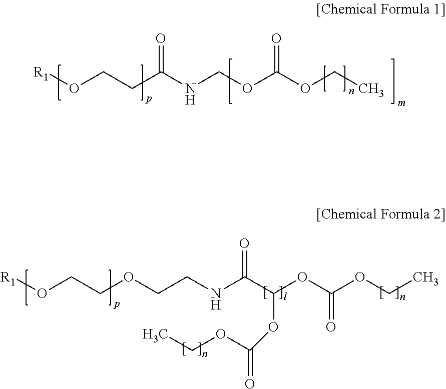
in Chemical Formula 1 or Chemical Formula 2,
R1 represents hydrogen, a C₁ to C₅ alkyl group, an amine group, a C₁ to C₅ alkylamine group, a carboxyl group, or a C₁ to C₅ alkylcarboxyl group,
p is an integer of 12 to 227,
n is an integer of 2 to 14,
m is an integer of 1 to 3, and
l is an integer of 4.

9. The method of claim 8, wherein the mixed solvent is a mixed solvent of methylene chloride and acetonitrile and a mixing ratio is 3 to 1:1 to 3.

10. The method of claim 8, wherein a molecular weight (Mn) of polyethylene glycol is 2,000 to 7,000.

11. The method of claim 8, wherein the micelle is synthesized in step 2 by dissolving the hydroxylated polyethylene glycol derivative and a compound represented by Chemical Formula 5 below in one or a mixed solvent of two or more selected from acetonitrile, methylene chloride, chloroform, and methanol and evaporating the solvent, and the compound represented by Chemical Formula 5 below is prepared by synthesizing a polyethylene glycol derivative by mixing polyethylene glycol and an alkyl chloroformate, and synthesizing a peptide-conjugated polyethylene glycol derivative by conjugating a nona-arginine (r9) peptide to the polyethylene glycol derivative: 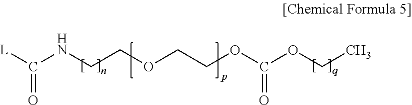
in Chemical Formula 5,
p is an integer of 12 to 227,
q is an integer of 2 to 14,
n is an integer of 0 to 5, and
L is a nona-arginine (r9) peptide.

12. A composition for fat reduction comprising the carbon dioxide-generating micelle according to claim 1.

13. The composition of claim 12, comprising a carbon dioxide-generating micelle including one or more compounds selected from the group consisting of a compound represented by Chemical Formula 1 below and a compound represented by Chemical Formula 2 below, and a compound represented by Chemical Formula 5 below: 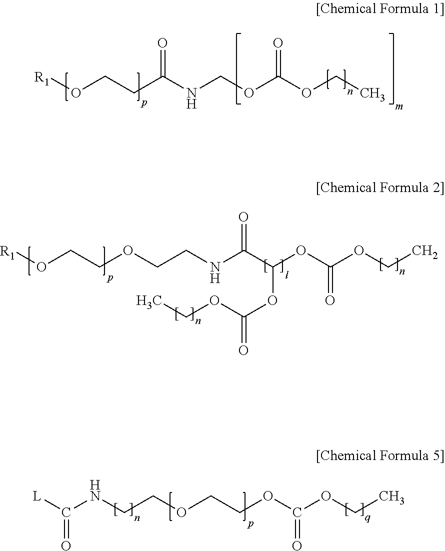
in Chemical Formula 1 or Chemical Formula 2,
R1 represents hydrogen, a C₁ to C₅ alkyl group, an amine group, a C₁ to C₅ alkylamine group, a carboxyl group, or a C₁ to C₅ alkylcarboxyl group,
p is an integer of 12 to 227,
n is an integer of 2 to 14,
m is an integer of 1 to 3, and
l is an integer of 4, and
in Chemical Formula 5,
p is an integer of 12 to 227,
q is an integer of 2 to 14,
n is an integer of 0 to 5, and
L is a nona-arginine (r9) peptide.

14. The composition of claim 12, which is used for local or intravenous injection.

15. A method for fat reduction in a subject in need thereof, the method comprising:
administering an effective amount of a composition comprising the carbon dioxide-generating micelle according to claim 1 capable of fat reduction in the body of a subject in need thereof.
